(19) United States
(12) Patent Application Publication
(10) Pub. No.: US 2014/0316247 A1
(43) Pub. Date: Oct. 23, 2014

(54) METHOD, APPARATUS, AND SYSTEM FOR TRACKING DEFORMATION OF ORGAN DURING RESPIRATION CYCLE

(71) Applicant: Samsung Electronics Co., Ltd., Suwon-si (KR)

(72) Inventors: Young-kyoo HWANG, Seoul (KR); Jung-bae KIM, Hwaseong-si (KR); Young-taek OH, Seoul (KR); Do-kyoon KIM, Seongnam-si (KR); Won-chul BANG, Seongnam-si (KR)

(73) Assignee: Samsung Electronics Co., Ltd., Suwon-si (KR)

(21) Appl. No.: 14/084,191

(22) Filed: Nov. 19, 2013

(30) Foreign Application Priority Data
Apr. 22, 2013 (KR) 10-2013-0044330

(51) Int. Cl. A61B 5/08; A61B 8/08

(52) U.S. Cl. CPC A61B 5/0806; A61B 8/08
USPC 600/411; 600/437; 600/427

(57) ABSTRACT

A method and apparatus of tracking a change in a region of interest in an subject according to respiration are provided. For example, an apparatus embodiment may include a model selector configured to select a model from among models of a region of interest of an subject generated to indicate a change in a region of interest during a respiration cycle of the subject, a respiration signal obtainer configured to obtaining a respiration signal of the region of interest by using ultrasound images including the region of interest obtained during the respiration cycle of the subject, and an information obtainer configured to obtain information regarding the region of interest at a time when the ultrasound images are obtained, from the selected model, by using the obtained respiration signal.

### CROSS-REFERENCE TO RELATED APPLICATIONS

This application claims the benefit of Korean Patent Application No. 10-2013-0044330 filed on Apr. 22, 2013, in the Korean Intellectual Property Office, the disclosure of which is incorporated herein in its entirety by reference.

### BACKGROUND

1. Field

The following description relates to methods, apparatuses, and systems for tracking deformation of organs during a respiration cycle.

2. Description of Related Art

A high-intensity focused ultrasound (HIFU) treatment is a method of removing and treating a tumor or another type of lesion by radiating HIFU to a tumor portion at a focus that is to be treated and causing a focal destruction or necrosis in a tumor tissue. The HIFU treatment accomplishes this task by causing ultrasound energy to be focused at a particular point within a patient's body. The focused ultrasound energy cauterizes that area of the patient's body, thereby destroying the cancerous tissue through a conversion of ultrasound energy to heat energy with a minimum of damage to healthy tissue.

A method of removing a lesion by using HIFU treats the tumor portion without directly cutting a human body and thus is a widely used treatment method. When HIFU is radiated into the lesion from the outside of the human body, a location of the lesion can change due to activity of the human body. For example, when a patient respires in surgery, the location of the lesion is changed by the respiration. In this example, if the patient has a tumor on his or her lungs, as the patient breathes the patient's lungs will deform as the lungs expand and shrink during the respiration process. Accordingly, a location (focus) to which HIFU is radiated in such a situation needs to be changed. If the HIFU is radiated to a fixed region, the HIFU will only fall upon the lesion some of the time, and at other times the HIFU may fall upon healthy areas of the patient, potentially injuring the patient. Such a method of radiating HIFU by tracking the lesion changed by the activity of the human body and using the information about the changing location of the lesion is a necessary approach in order to successfully treat lesions whose locations change during respiration.

Locations of organs are changed by respiration, and additionally, shapes of organs are changed. Additionally, changes in locations and shapes of organs due to respiration are closely related to each other, as the changes in locations and shapes of organs are due to positional changes and deformations of organs during respiration. For example, the lungs change shape as they inflate and deflate during respiration.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Provided are organ change tracking methods and devices and systems during a respiration cycle. Also provided are computer-readable recording media on which a program for carrying out the method on a computer is recorded.

Additional aspects will be set forth in part in the description which follows and, in part, will be apparent from the description, or may be learned by practice of the presented embodiments.

In one general aspect, a method of tracking a change in a region of interest of a subject according to respiration includes generating models indicating a change in a location or a shape of the region of interest of the subject during a respiration cycle of the subject by using external images including the region of interest obtained at two times of the respiration cycle of the subject, selecting a model having the highest similarity to 3D ultrasound images including the region of interest obtained at one or more times of the respiration cycle of the subject, obtaining a respiration signal of the region of interest by using 2D ultrasound images including the region of interest obtained during the respiration cycle of the subject, and obtaining information regarding the region of interest at a time when the 2D ultrasound images are obtained, from the selected model, by using the obtained respiration signal.

The external images may be magnetic resonance (MR) images or computed tomography (CT) images.

The obtaining of the respiration signal may include selecting an object from which the respiration signal is to be obtained from the 2D ultrasound images, selecting a specific window from windows disposed in a location indicating the selected object from the 2D ultrasound images, and generating the respiration signal by using motion information of the object included in the specific window, wherein the windows have different sizes, directions, and locations disposed on the 2D ultrasound images to obtain the motion information of the object according to the respiration.

The respiration signal may be a signal indicating a displacement of the region of interest that changes according to the subject's respiration.

The object may be an object having a brightness value exceeding a threshold value among organs included in the 2D ultrasound images.

The selecting of the object may include segmenting information regarding a boundary line of the object from the 2D ultrasound images, and obtaining a center line of the object by using the segmented information regarding the boundary line, wherein the specific window is selected by placing the windows on the obtained center line.

The specific window may be selected by using at least one of noise information of the 2D ultrasound images or the motion information of the object.

The two times may be maximum inspiration time and maximum expiration time of the subject.

The generating of the models may include segmenting surface information of tissues included in the external images obtained at the maximum inspiration time and the external images obtained at the maximum expiration time, and performing interpolation by using the segmented surface information.

The selecting of the model may include segmenting surface information of tissues included in the 3D ultrasound images, matching the models and the 3D ultrasound images by using the segmented surface information, and calculating similarity between the models and the 3D ultrasound images by using the matching images and selecting a model having the highest similarity between the models and the 3D ultrasound images by using the calculated similarity.

The obtaining of the information may include obtaining information regarding the region of interest by using at least one of a displacement value of the region of interest at the time when the 2D ultrasound images are obtained and maximum and minimum values of the displacement value of the region of interest included in the selected model, wherein the time when the 2D ultrasound images are obtained comprises a time of the respiration cycle of the subject.

The method may further include generating ultrasound that is to be radiated to the lesion tissue by using the obtained information regarding the region of interest.

In another general aspect, there is provided a non-transitory computer-readable storage medium storing a program for tracking a change in a region of interest, the program comprising instructions for causing a computer to carry out the method of the embodiment described above.

In another general aspect, an apparatus for tracking a change in a region of interest of a subject according to respiration includes a model generator configured to generate models indicating a change in a location or a shape of the region of interest of the subject during a respiration cycle of the subject by using external images including the region of interest obtained at two times of the respiration cycle of the subject, a model selector configured to select a model having the highest similarity between the models and 3D ultrasound images including the region of interest obtained at one or more times of the respiration cycle of the subject, a respiration signal obtainer configured to obtain a respiration signal of the region of interest by using 2D ultrasound images indicating the region of interest obtained during the respiration cycle of the subject, and an information obtainer configured to obtain information regarding the region of interest at a time when the 2D ultrasound images are obtained, from the selected model, by using the obtained respiration signal.

The external images may be magnetic resonance (MR) images or computed tomography (CT) images.

The apparatus may provide that the respiration signal obtainer is configured to select an object from which the respiration signal is to be obtained from the 2D ultrasound images, configured to select a specific window from windows disposed in a location indicating the selected object from the 2D ultrasound images, and configured to generate the respiration signal by using motion information of the object included in the specific window, wherein the windows have different sizes, directions, and locations disposed on the 2D ultrasound images to obtain the motion information of the object according to the respiration.

The apparatus may provide that the object is selected by segmenting information regarding a boundary line of the object from the 2D ultrasound images, and obtaining a center line of the object by using the segmented information regarding the boundary line, wherein the specific window is selected by placing the windows on the obtained center line.

The apparatus may provide that the model generator is configured to segment surface information of tissues included in the external images obtained at two times of the respiration cycle of the subject and configured to perform interpolation by using the segmented surface information, wherein the two times are maximum inspiration time and maximum expiration time of the subject.

The apparatus may provide that the model selector is configured to segment surface information of tissues included in the 3D ultrasound images, configured to match the models and the 3D ultrasound images by using the segmented surface information, configured to calculate similarity between the models and the 3D ultrasound images by using the matching images, and configured to select a model having the highest similarity between the models and the 3D ultrasound images by using the calculated similarity.

The apparatus may further include an ultrasound generator configured to generate diagnosis ultrasound that is to be radiated to the lesion tissue by using the obtained information regarding the region of interest.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram illustrating an imaging processing apparatus according to an example embodiment.

FIG. 2 is a diagram illustrating an example of operating a model generator.

FIG. 3 is a diagram illustrating another example of operating a model generator.

FIGS. 4A through 4D are diagrams illustrating an example in which a respiration signal obtainer selects an object from which a respiration signal is to be obtained from a 2D ultrasound image.

FIG. 5 is a diagram illustrating an example of windows disposed on a 2D ultrasound image.

FIGS. 6A and 6B are diagrams illustrating an example in which a respiration signal obtainer selects a specific window.

FIGS. 7A through 7C are graphs illustrating an example of a respiration signal obtained by a respiration signal obtainer.

FIG. 8 is a block diagram illustrating another imaging processing apparatus, according to another embodiment.

FIG. 9 is a diagram illustrating an environment in which an organ change tracking system is used.

FIG. 10 is a flowchart illustrating a method of tracking a change of an organ performed by an image processing apparatus, according to an example embodiment.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The present invention now will be described more fully hereinafter with reference to the accompanying drawings, in which elements of the invention are shown. The present invention may, however, be embodied in many different forms and should not be construed as limited to the exemplary embodiments set forth herein. Rather, these embodiments are provided so that this disclosure will be thorough and complete, and will fully convey the scope of the present invention to one of ordinary skill in the art. Numerous modifications and adaptations will be readily apparent to one of ordinary skill in this art from the detailed description and the embodiments without departing from the spirit and scope of the present invention. As used herein, the term “and/or” includes any and all combinations of one or more of the associated listed items. Expressions such as “at least one of,” when preceding a list of elements, modify the entire list of elements and do not modify the individual elements of the list.

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

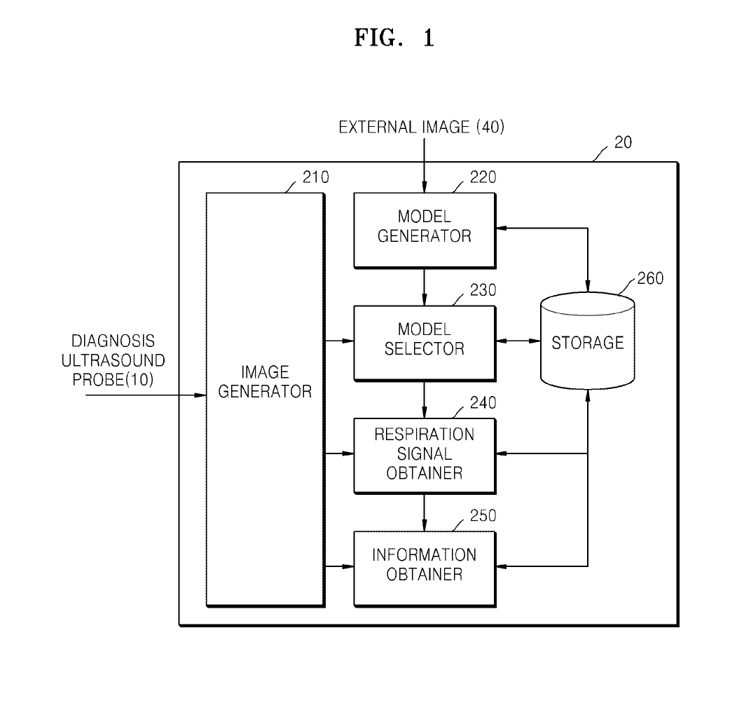

FIG. 1 is a block diagram illustrating an imaging processing apparatus 20, according to an example embodiment.

Referring to FIG. 1, the imaging processing apparatus 20 includes an image generator 210, a model generator 220, a model selector 230, a respiration signal obtainer 240, an information obtainer 250, and a storage 260. The imaging processing apparatus 20, in some embodiments, may further include general-purpose elements other than the elements shown in FIG. 1. Additionally, alternative elements that perform the operation of the imaging processing apparatus 20 may be used instead of the elements shown in FIG. 1.

Also, in some embodiments, each of the image generator 210, the model generator 220, the model selector 230, the respiration signal obtainer 240, the information obtainer 250, and the storage 260 of the imaging processing apparatus 20 of FIG. 1 may correspond to one or more processors. In examples, a processor includes an array of logic gates, or a combination of a general-purpose microprocessor and a program that is executed by the microprocessor. Alternatively, it would be understood by one of ordinary skill in the art that the processor includes any of other types of hardware that participate in processing information for the imaging processing apparatus 20.

The image generator 210 may receive pulse signals from a diagnosis ultrasound probe 10 and may generate a 2D ultrasound image or a 3D ultrasound image with respect to a region of interest 30, based on the pulse signals from the diagnosis ultrasound probe 10. In this regard, a lesion tissue may be included in the image of the region of interest, 30.

According to an aspect, the 3D ultrasound image is used to match a model indicating a change in a location or a shape of the region of interest 30 generated by the model generator 220 that will be described later. The 2D ultrasound image is used to extract a respiration signal of the region of interest 30 and select a model corresponding to a change in the region of interest 30 according to a current respiration state of a subject (for example, a patient) in real time. For example, as the subject respires, the respiration process may be modeled so that each stage in the respiration process corresponds to an appropriate model.

In an embodiment, the 3D ultrasound image may be obtained before surgery is performed on the subject and the 2D ultrasound image may be obtained several times before and when surgery is performed on the subject. However, implementation is not limited thereto, and more or less 3D or 2D images may be obtained at different stages of the treatment process appropriately in differing embodiments.

The diagnosis ultrasound probe 10 may radiate diagnostic ultrasound to the region of interest 30 of the subject, and obtain a reflected ultrasound signal. More specifically, if diagnosis ultrasound probe 10 radiates diagnostic ultrasound in the range of 2 and 18 MHz to the region of interest 30 of the subject, the diagnostic ultrasound is partially radiated from layers of other tissues. However, some embodiments may use diagnostic ultrasound that is slightly above or below this range. The diagnostic ultrasound is reflected at a portion within the region of interest 30 having a density change, for example, blood cells of blood plasma, small structures of organs, etc. Such reflected diagnostic ultrasound vibrates a piezoelectric converter of the diagnosis ultrasound probe 10. The piezoelectric converter outputs electrical pulses according to the vibrations resulting from the received reflected diagnostic ultrasound.

However, the diagnosis ultrasound probe 10 may directly generate an ultrasound image representing the region of interest 30 based on the electrical pulses. When the diagnosis ultrasound probe 10 directly generates the ultrasound image, the diagnosis ultrasound probe 10 transmits information regarding the generated ultrasound image to the image generator 210.

Meanwhile, when the image generator 210 generates the ultrasound image, the diagnosis ultrasound probe 10 transmits electrical pulses to the image generator 210.

The 2D ultrasound image or the 3D ultrasound image with respect to the region of interest 30 may be generated by one diagnosis ultrasound probe 10 or a plurality of diagnosis ultrasound probes 10. More specifically, the diagnosis ultrasound probe 10 for generating the 2D ultrasound image and the diagnosis ultrasound probe 10 for generating the 3D ultrasound image may be separately provided. Hence, one or more diagnosis ultrasound probes 10 may interact to produce 2D ultrasound image(s) and one or more diagnosis ultrasound probes 10 may interact to produce 3D ultrasound image(s), and the one or more diagnosis ultrasound probes 10 may or may not be shared when generating 2D and 3D ultrasound image(s).

For example, when one diagnosis ultrasound probe 10 is used to generate the 3D ultrasound image, the image generator 210 accumulates 2D cross-sectional images generated by the diagnosis ultrasound probe 10 and generates the 3D ultrasound image indicating the region of interest 30 in a 3D manner. An example of such a 3D manner is a multiplanar reconstruction (MPR) method. However, implementations are not limited to such a method of generating the 3D ultrasound image performed by the image generator 210, and other embodiments may use other appropriate methods.

The model generator 220 may generate models indicating a change in a location or a shape of the region of interest 30 during a single respiration cycle of the subject by using magnetic resonance (MR) images or computed tomography (CT) images including the region of interest 30 of the subject obtained at two times during the single respiration cycle. More specifically, in an embodiment, the two times during the single respiration cycle at which the MR or CT images are obtained are a maximum inspiration time and a maximum expiration time. These times represent the limits of the respiration process, and any location and shape of the region of interest 30 should fall between these two images. However, if additional, intermediate MR or CT images are available, the additional images may be incorporated into the modeling process as well.

In this context, the model generator 220 generates the models as a preparation step before surgery is performed on the subject. For example, as one of a set of surgery preparation operations performed on the subject, the model generator 220 may generate the models indicating the change in the location or the shape of the region of interest 30 during the single respiration cycle of the subject, based on the images of the extremes of the single respiration cycle, as discussed above.

A model generation method performed by the model generator 220 will now be further described.

In an example embodiment, the model generator 220 segments surface information regarding tissues included in MR or CT images obtained at a maximum inspiration time during a respiration cycle and MR or CT images obtained at a maximum expiration time. In this regard, the MR or CT images are images including anatomical information regarding tissues included in the region of interest 30, and in some embodiments, a depiction of a lesion tissue is included in the MR or CT images of the tissues. The model generator 220 generates the models by performing interpolation using the segmented surface information.

In this example, the desired model includes a set of images indicating the changes in the location or the shape of the region of interest 30 during the single respiration cycle of the subject. The model generator 220 generates models with respect to at least two respiration cycles. More specifically, the model generator 220 generates the models with respect to the at least two respiration cycles by repeating an operation of generating the models of the region of interest 30 during the single respiration cycle with respect to at least two respiration cycles. That is, in some embodiments, the model generator 220 generates a first model with respect to a first respiration cycle of the subject and generates a second model with respect to a second respiration cycle. In an embodiment, one of the cycles is inspiration, and the other cycle is expiration. More information about the modeling process is provided, below.

The model generator 220 may receive an MR or CT image, hereinafter referred to as an external image 40, directly from an external capturing apparatus or the storage 260 in which images are stored.

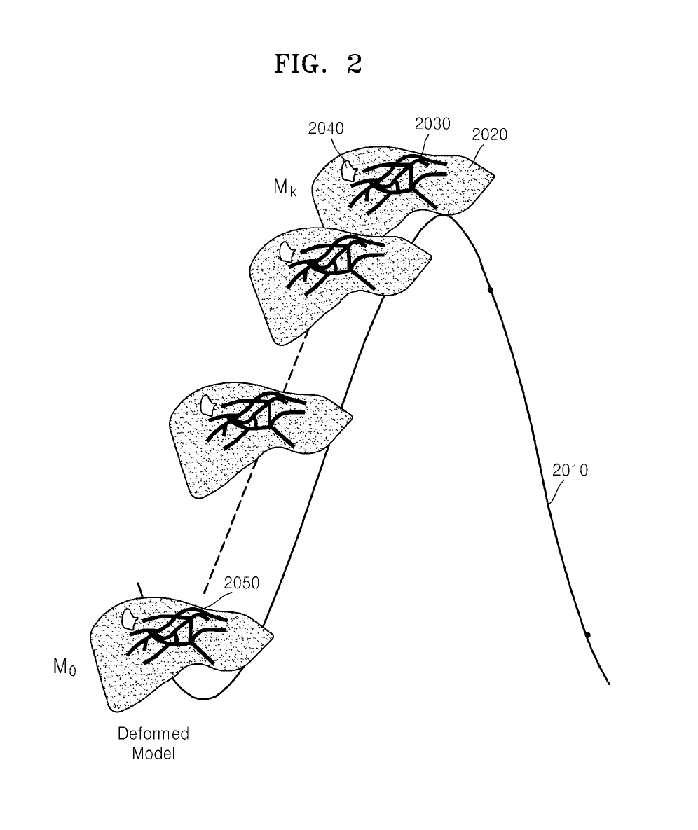

FIG. 2 is a diagram illustrating an example of operating the model generator 220, according to an embodiment.

The model generator 220 may segment surface information of tissues included in the region of interest 30 of the external image 40 obtained at the maximum inspiration time Mₖ during a respiration cycle 2010 of a subject. For example, provided that the region of interest 30 of the external image 40 is a liver 2020 of the subject, the model generator 220 segments a surface of the liver 2020 and a surface of a blood vessel 2030 distributed in the liver 2020. If a lesion 2040 is present in the liver 2020 of the subject, the model generator 220 segments a surface of the lesion 2040. In this regard, in some embodiments the surface is defined as a boundary line of a tissue.

The model generator 220 may segment surface information of tissues included in the region of interest 30 of the external image 40 obtained at the maximum expiration time M₀ during the respiration cycle 2010 of the subject in the same manner as described above.

In this regard, the method of segmenting the surface information of the tissues included in the external image 40 performed by the model generator 220 is performed using approaches known to one of ordinary skill in the art, and thus a further description is omitted here for conciseness.

Thereafter, the model generator 220 performs interpolation by using the segmented surface information. For example, the model generator 220 may perform interpolation using Bezier curve interpolation. However, other methods of interpolation may be used in other embodiments.

More specifically, the model generator 220 performs interpolation between the segmented surface information by identifying shapes in the segmented surfaces that correspond to each other. For example, the model generator 220 may perform interpolation using information regarding the surface of the blood vessel 2030 segmented from an image at the maximum inspiration time Mₖ and information regarding the surface of the blood vessel 2030 segmented from an image at the maximum expiration time M₀.

The model generator 220 performs interpolation on regions corresponding to each other in the two images by using the same method described above, thereby generating models indicating changes in locations and shapes of organs or lesions included in a region of interest during the respiration cycle 2010. In this regard, the method of performing interpolation, for example, Bezier curve interpolation, performed by the model generator 220 of FIG. 1 may be performed using approaches known to one of ordinary skill in the art, and thus a further description is omitted here for conciseness.

Referring to FIG. 1, the model generator 220 transmits the generated models to the storage 260, where the generated models are stored for later retrieval and usage. Meanwhile, as described above, the model generator 220 generates two or more models with respect to respiration cycles of the subject, such as inspiration and expiration, and transmits the generated models to the storage 260. In this regard, in some embodiments the generated models may include images in a mesh shape indicating the surface information of the tissues included in the region of interest 30.

The model selector 230 selects a model having the highest similarity between 3D ultrasound images and the models including the region of interest 30 obtained at one or more times during a respiration cycle of the subject from among the models. Thus, the role of the model selector 230 is to establish a correspondence between the pre-existing models and the ultrasound images obtained in real-time. In this regard, one or more times may refer to the maximum inspiration time and/or the maximum expiration time during the respiration cycle of the subject.

The subject's respiration is usually normal in a comfortable range in surgery. The subject may be a patient. The subject is unlikely to repeat a maximum inspiration and a maximum expiration during surgery itself, because the respiration will usually occur somewhere in between the two maxima. A 2D ultrasound image as will be described later that is obtained in surgery performed on the subject, may include information regarding a change in the region of interest 30 according to a usual respiration process that is characteristic of the subject. Therefore, 3D ultrasound images obtained before the surgery is performed on the subject may be generated at the maximum inspiration time and/or the maximum expiration time during the respiration cycle of the subject, so as to define a range of respiration when modeling the respiration that actually occurs. That is, change information of the region of interest 30 included in 3D ultrasound images may be made to correspond to change information of the region of interest 30 included in 2D ultrasound images by including a range of changes in a model. In this way, the change information provides information regarding changes in a location and a shape of the region of interest 30 according to respiration based on ultrasound information.

Concurrently, change information of the region of interest 30 included in a model generated based on the external image 40 may include the change information of the region of interest 30 included in the 3D ultrasound images since the model selector 230 may select a model having the highest similarity between the 3D ultrasound images and the generated models from the generated models. That is, a condition in which the change in the location and the shape of the region of interest 30 is the same or smaller than a threshold may be satisfied by comparing the maximum inspiration and/or the maximum expiration used to generate the 3D ultrasound images with the maximum inspiration and/or the maximum expiration used to generate the models.

For example, the model selector 230 may select a model having the highest similarity between the 3D ultrasound images transmitted from the image generator 210 and models stored in the storage 260 from the models. In this context, the 3D ultrasound images may be obtained before surgery is performed on the subject, and the operation of selecting the model having the highest similarity between the 3D ultrasound images and the models performed by the model selector 230 may be performed before surgery is performed on the subject. By making such a selection, embodiments may choose a model such that the information provided by the external image 40 is coordinated with the ultrasound information to help model changes in the location and shape of the region of interest 30.

An example of selecting the model having the highest similarity between the 3D ultrasound images and the models from the models performed by the model selector 230 will now be described.

The model selector 230 segments surface information of tissues included in the 3D ultrasound images. In this regard, a surface may means a boundary line of a tissue. A method of segmenting the surface information of the tissues included in the 3D ultrasound images may be the same as described above, being an existing technique for the performance of this task.

The model selector 230 matches the models and the 3D ultrasound images by using the segmented surface information. In an embodiment, the model selector 230 performs matching by using an iterative closest point (ICP) algorithm. The ICP algorithm is an algorithm used for rotation, parallel movement, and scaling of other images with respect to one image to align targets included in a plurality of images. The ICP algorithm is an approach known to one of ordinary skill in the art, and thus a further description is omitted here for conciseness. Alternatively, other algorithms that match the models and 3D ultrasound images may be used in different embodiments.

The model selector 230 calculates similarities between the models and the 3D ultrasound images by using the matching images, and selects the model having the highest similarity therebetween from the models by using the calculated similarities. In this process, similarities may be calculated by calculating an average distance between points of closest approach of shapes included in the matching images.

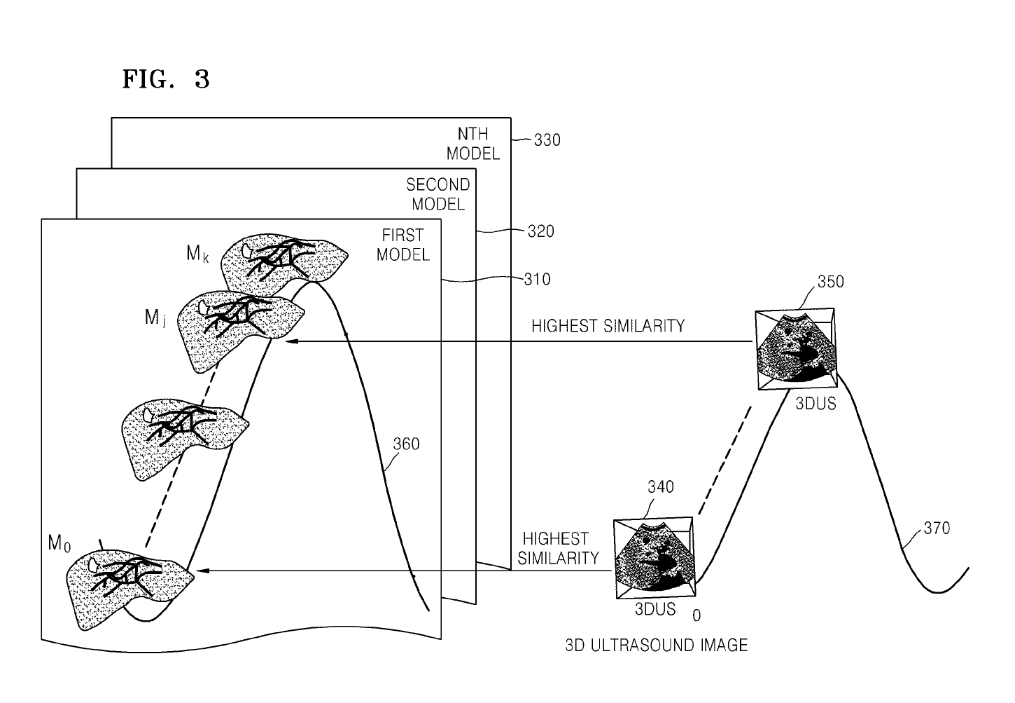

FIG. 3 is a diagram illustrating another example of operating the model generator 220, according to an embodiment.

As described above, the model generator 220 matches each of models 310 through 330 and a 3D ultrasound image 340 or 350 and calculates a similarity therebetween. The model generator 220 may select the model 310 having the highest similarity therebetween from the models 310 through 330. Reference numerals 360 and 370 of FIG. 3 denote respiration cycles of a subject.

Referring to FIG. 1, the model generator 220 transmits information regarding the selected model 310 to the storage 260. For example, the model selector 230 may identify a selected model and other models by separately marking the model selected by using the above-described method from models stored in the storage 260.

The respiration signal obtainer 240 obtains a respiration signal of the region of interest 30 by using 2D ultrasound images indicating the region of interest 30 obtained during a respiration cycle of the subject. For example, the respiration signal obtainer 240 may obtain the respiration signal of the region of interest 30 by using 2D ultrasound images transmitted from the image generator 210. In this context, the respiration signal is a signal indicating a displacement of the region of interest 30 that changes according to the subject's respiration and may be obtained during surgery performed on the subject.

More specifically, the respiration signal obtainer 240 may select an object from which the respiration signal is to be obtained from a 2D ultrasound image obtained before surgery is performed on the subject. Thereafter, the respiration signal obtainer 240 may select a specific window from among windows disposed in various locations indicating the object selected from the 2D ultrasound image. In this regard, the windows are disposed on 2D ultrasound images and have different sizes, directions, and locations to obtain motion information regarding an object according to changes that occur during respiration. The specific window is a window that most accurately expresses the motion information regarding the object from the candidate windows.

Thereafter, the respiration signal obtainer 240 may place the specific window on the 2D ultrasound image obtained in real time during the surgery performed on the subject. The respiration signal obtainer 240 obtains the respiration signal by using the motion information regarding the object displayed on the specific window.

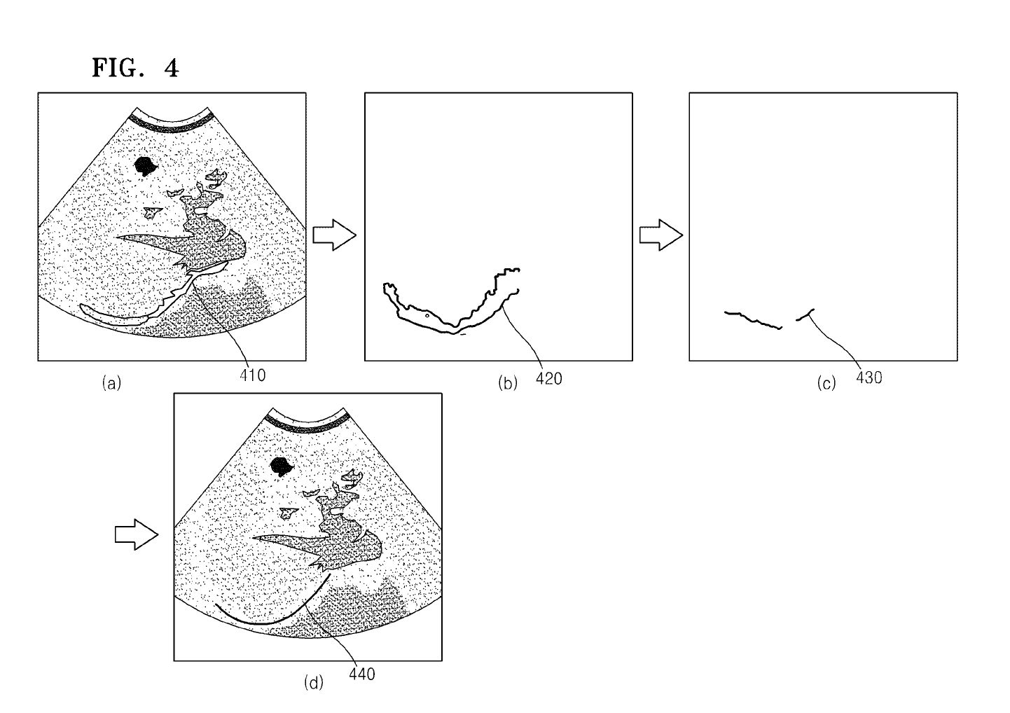

FIGS. 4A through 4D are diagrams illustrating an example in which the respiration signal obtainer 240 selects an object from which a respiration signal is to be obtained from a 2D ultrasound image, according to an embodiment.

As described above, the respiration signal obtainer 240, in some embodiments performs an operation of selecting the object from which the respiration signal is to be obtained before surgery is performed on a subject.

Referring to FIG. 4A, the respiration signal obtainer 240 selects an object 410 from organs included in the region of interest 30 of the 2D ultrasound image. In this regard, the object 410 may refer to an organ having a brightness value exceeding a threshold value from the organs included in the 2D ultrasound image. More specifically, in this example the respiration signal obtainer 240 selects a region, in which the respiration signal is strongly generated, as the object. For example, the region may be derived from information including noise of an ultrasound image, detected abdominal fat of the subject, for example, a patient, cirrhosis, and a sonic shadow.

For example, when lung, liver, and diaphragm are included in the organs included in the 2D ultrasound image, the respiration signal obtainer 240 in an example may select the diaphragm as the object 410 by using a property of the diaphragm as having a relatively bright line in the 2D ultrasound image.

Referring to FIG. 4B, the respiration signal obtainer 240 segments information regarding a boundary line 420 of the object 410 from the 2D ultrasound image. For example, the respiration signal obtainer 240 may obtain coordinate information of a point of the 2D ultrasound image at which brightness rapidly changes, and may extract a location having the largest frequency value as the boundary line 420 by using an appropriate technique, such as a discrete time Fourier transform (DTFT).

For another example, if the respiration signal obtainer 240 receives information regarding some boundary points included in an ultrasound image from a user through an interface (not shown), the respiration signal obtainer 240 may extract the boundary line 420 based on the boundary points in the same manner as described above.

Referring to FIG. 4C, the respiration signal obtainer 240 obtains a center line 430 of the object 410 by using the previously obtained segmented information regarding the boundary line 420. For example, the respiration signal obtainer 240 in one embodiment may obtain the center line 430 by using a distance transform.

In this regard, the distance transform means calculation of a distance from a pixel on an image to an object closest to the pixel. More specifically, the respiration signal obtainer 240 may calculate a distance from each of all of the pixels included in the extracted center line 430 to the center line 430 closest to the pixel.

Thereafter, the respiration signal obtainer 240 may obtain the center line 430 of the object 410 by connecting pixels having the largest distance value. Specific algorithms of the distance transform are known to one of ordinary skill in the art, and thus a further description thereof is omitted here for conciseness.

Referring to FIG. 4D, the respiration signal obtainer 240 may obtain a shape 440 of the object 410 through an appropriate method, such as polynomial fitting, by using the center line 430 of the object 410. In this regard, specific algorithms such as polynomial fitting are known to one of ordinary skill in the art, and thus a further description thereof is omitted here for conciseness.

Referring to FIG. 1, the respiration signal obtainer 240 selects the specific window from the windows disposed in the location indicating the selected object in the 2D ultrasound image. In this regard, the respiration signal obtainer 240 may perform the operation of selecting the specific window, as discussed above, before surgery is performed on the subject.

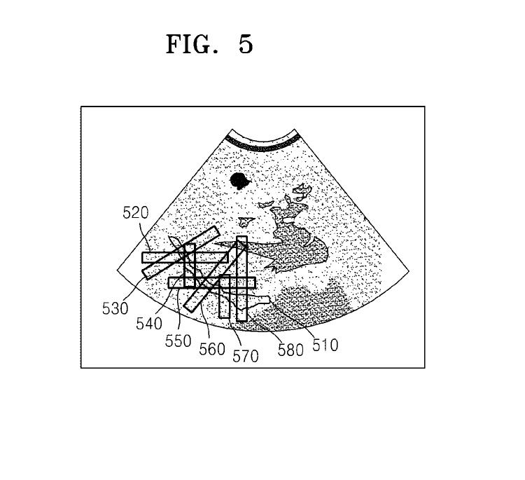

FIG. 5 is a diagram illustrating an example of windows 520 through 580 disposed on a 2D ultrasound image, according to an embodiment.

Referring to FIG. 5, the respiration signal obtainer 240 places the windows 520 through 580 on a shape 510 of an object in the 2D ultrasound image. In some embodiments, the windows 520 through 580 have different sizes, directions, and locations. However, the windows 520 through 580 are not necessarily limited to windows having different sizes, directions, and locations, and other embodiments may include windows with overlap or duplication.

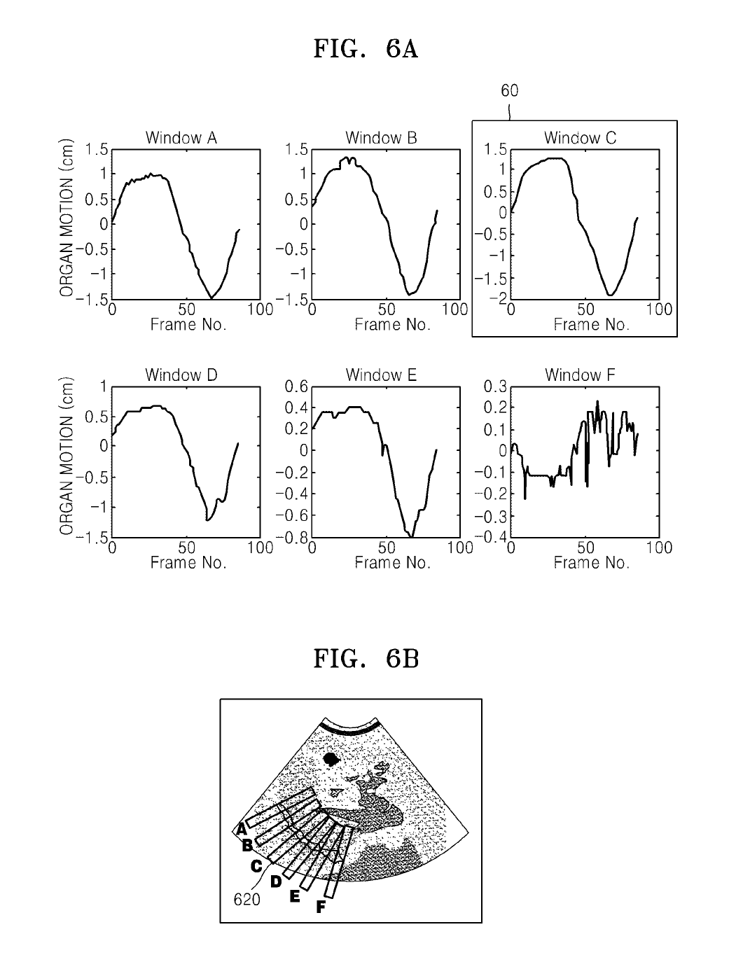

FIGS. 6A and 6B are diagrams illustrating an example in which the respiration signal obtainer 240 selects a specific window, according to an embodiment.

Referring to FIG. 6A, the respiration signal obtainer 240 obtains a respiration signal for each of windows A through F, shown in FIG. 6B, disposed on a shape of an object in a 2D ultrasound image.

In FIG. 6A, a graph shows the respiration signal for each of windows A through F. In this regard, the respiration signal that is graphed is a signal indicating a displacement of the region of interest 30 that changes according to a subject's respiration.

Whenever a subject respires, locations of organs of the subjects change. For example, respiration includes inhalation and exhalation of gas, and blood flow changes during respiration as well. Thus, a location of the object selected by the respiration signal obtainer 240 also changes whenever the subject respires. Therefore, if motions of objects included in the windows A through F disposed on the 2D ultrasound image obtained during a respiration cycle of the subject are observed, the displacement of the region of interest 30 that changes according to the subject's respiration may be known.

The respiration signal obtainer 240 may obtain the respiration signal of an object included in each of the windows A through F and may select a respiration signal 610 that most accurately expresses the subject's respiration from the respiration signals. To do so, the respiration signal obtainer 240 selects a window 620, from which the selected respiration signal 610 is selected, as the specific window.

The respiration signal obtainer 240 may select the specific window by using at least one of motion information of objects and noise information of the 2D ultrasound image. For example, in one case the respiration signal obtainer 240 may select a window having large motion and small noise of the objects included in the windows A through F as the specific window.

The respiration signal obtainer 240 may calculate motion S₁ᵢ of the objects included in the windows A through F according to Equation 1 below.

S₁ᵢ=Max(Fᵢ)−Min(Fᵢ)

In Equation 1 above, Fᵢ is defined as Fᵢ=[m(0), . . . ,m(t)]^{T} and denotes a location vector of an object included in an i^{th} window disposed in 2D ultrasound images. m(t) denotes a location of an object included in a t^{th} image of 2D ultrasound images.

The respiration signal obtainer 240, in an embodiment, may calculates noise S₂ᵢ included in the windows A through F according to Equation 2 below.

$${S}_{2 i} = \frac{{\sum }_{k}^{ } {( {F}_{k}^{″} )}^{2}}{\# \vert {F}_{i}^{″} \vert} - ( \frac{{\sum }_{k}^{ } {F}_{ik}^{″}}{\# \vert {F}_{i}^{″} \vert} ) \qquad Equation 2$$

In Equation 2 above, F″ᵢ denotes a second derivative of Fᵢ. That is, F″ᵢ denotes acceleration regarding a motion of the object included in the i^{th} window disposed in 2D ultrasound images. #|F″ᵢ| denotes a cardinality of F″ᵢ.

The respiration signal obtainer 240 may calculate a score Wᵢ of the i^{th} window disposed in 2D ultrasound images by substituting the motion S₁ᵢ of the objects and the noise S₂ᵢ into Equation 3 below.

$${W}_{i} = p \cdot \frac{{S}_{1 i}}{\vert \sum {S}_{1 i} \vert} + ( 1 - p ) \cdot \frac{{S}_{2 i}}{\vert \sum {S}_{2 i} \vert} \qquad Equation 3$$

In Equation 3 above, p denotes a weight regarding the motion S₁ᵢ of the objects and the noise S₂ᵢ and satisfies pε[0,1]. That is, p is a variable defining which one has more weight when deriving a score based on the motion S₁ᵢ of the objects and the noise S₂ᵢ when the respiration signal obtainer 240 selects the specific window. In some embodiments the respiration signal obtainer 240 may automatically determine p, and in other embodiments a user may designate p as a certain value through an interface (not shown).

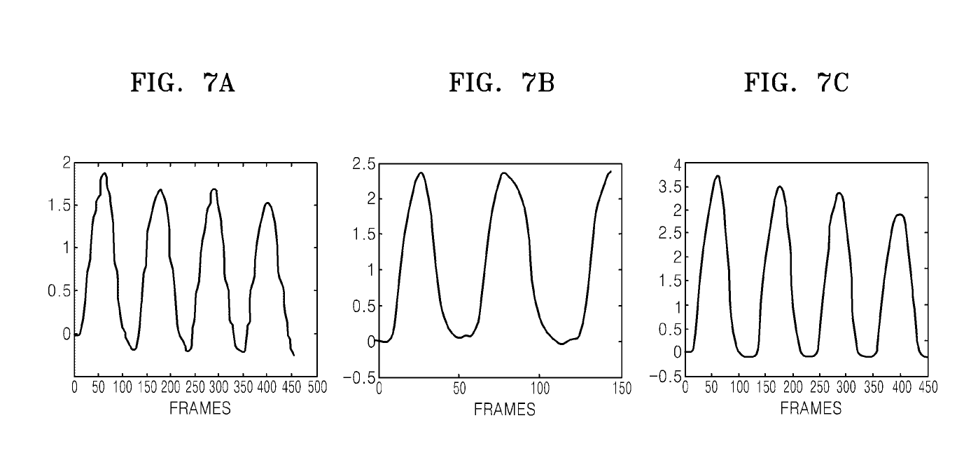

FIGS. 7A through 7C are graphs illustrating an example of a respiration signal obtained by the respiration signal obtainer 240, according to an embodiment.

In the graphs of FIGS. 7A through 7C, a horizontal axis corresponds to image frames constituting each of the 2D ultrasound images, and a vertical axis corresponds to a displacement of an object in a specific window. That is, 2D ultrasound images are images generated during the subject's respiration, and thus the horizontal axis is also regarded as a time flow. Thus, the graphs of FIGS. 7A through 7C are also regarded as a displacement of the region of interest 30 that changes according to the time flow.

In an example, the respiration signal obtainer 240 generates a displacement of an object included in a specific window that changes according to the time flow in a lookup table. For example, the respiration signal obtainer 240 assigns a number to each index included in a selected mode and may generate the lookup table including the assigned number of each index and a location of an object included in an index corresponding to the assigned number. The respiration signal obtainer 240 transmits and stores the generated lookup table to and in the storage 260.

The respiration signal may be obtained by using a 2D ultrasound image obtained in real time during surgery performed on a subject. For example, the respiration signal obtainer 240 performs an operation of selecting an object from which a respiration signal is to be obtained and selects a specific window by using a 2D ultrasound image obtained before surgery is performed on the subject. Meanwhile, the respiration signal obtainer 240 may perform an operation of extracting the respiration signal by using the 2D ultrasound image obtained in real time during surgery performed on the subject.

Referring to FIG. 1, the respiration signal obtainer 240 transmits information regarding the obtained respiration signal to the information obtainer 250.

The information obtainer 250 obtains information regarding a region of interest 30 at a time when the 2D ultrasound image is obtained from the selected model by using the obtained respiration signal. In this context, the time when the 2D ultrasound image is obtained is a time of a respiration cycle of the subject. For example, the information obtainer 250 obtains the information regarding the region of interest 30 by using the respiration signal transmitted from the respiration signal obtainer 240, the 2D ultrasound images transmitted from the image generator 210, and the model transmitted from the storage 260 and selected by the model selector 230.

An operation of obtaining the information regarding the region of interest 30 by using one of the 2D ultrasound images performed by the information obtainer 250 will now be described. The information obtainer 250 may obtain the information regarding the region of interest 30 during one respiration cycle by applying the operation that will be described later to the other 2D ultrasound images generated during one respiration cycle. In this regard, the information regarding the region of interest 30 is information regarding changes in locations and shapes of organs included in the region of interest 30. The 2D ultrasound images used by the information obtainer 250 are images obtained in real time during surgery performed on the subject.

In embodiments, the information obtainer 250 may obtain the information regarding the region of interest 30 by using at least one of a displacement value of the region of interest 30 and maximum and minimum values of the displacement value of the region of interest 30 included in the selected model.

As an example, provided that the information obtainer 250 uses an i^{th} image among the 2D ultrasound images to obtain the information regarding the region of interest 30, the information obtainer 250 may obtain a model index k corresponding to the i^{th} image from the selected model according to Equation 4 below.

$$k = round ( \frac{{S}_{i} - min ( RRS ) \cdot N}{max ( RRS ) - min ( RRS )} ) \qquad Equation 4$$

In Equation 4 above, Sᵢ denotes a location of an object included in the i^{th} image among the 2D ultrasound images, and N denotes a number of indices constituting the selected model.

Referring to FIG. 3, provided that the model selected by the model selector 230 needs indices from M₀ to Mₘ from indices constituting the first model 310, N may denote (m+1) in Equation 4 above.

Referring to FIG. 1, max(RRS) in Equation 4 above denotes the location of the region of interest 30 at the maximum inspiration appearing in 3D ultrasound images, and min(RRS) denotes the location of the region of interest 30 at the maximum expiration appearing in 3D ultrasound images. If the location of the region of interest 30 is normalized as a number from 0 to 1, in an example, max(RRS) may be 1, and min(RRS) may be 0.

As another example, the information obtainer 250 may obtain the model index k by using the lookup table stored in the storage 260. More specifically, provided that the information obtainer 250 uses the i^{th} image among the 2D ultrasound images to obtain the information regarding the region of interest 30, the information obtainer 250 obtains the model index k corresponding to the i^{th} image by using at least one relationship between locations of an object and each model index recorded in the lookup table.

The information obtainer 250 obtains information regarding the region of interest 30 corresponding to the index k from the selected model. More specifically, the information obtainer 250 obtains information regarding a location and shape of the region of interest 30 corresponding to the index k from the selected model. In an embodiment, the information obtainer 250 may also obtain information regarding the region of interest 30 during one respiration cycle by applying the above-described operations to other 2D ultrasound images generated during one respiration cycle.

As described above, the information obtainer 250 obtains the information regarding the region of interest 30 from the model, thereby obtaining an image regarding the region of interest 30 in real time during surgery. Changes in organs are tracked by using features clearly identified from ultrasound images, thereby managing noise. Changes in organs of a patient may also be accurately tracked, using these techniques.

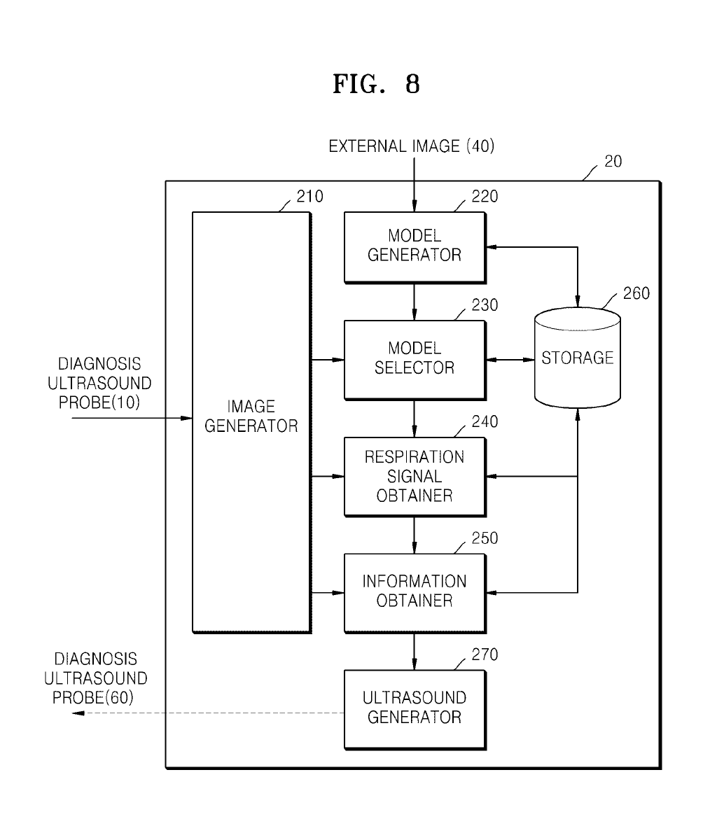

FIG. 8 is a block diagram illustrating the imaging processing apparatus 20, according to another embodiment.

Referring to FIG. 8, the imaging processing apparatus 20 includes the image generator 210, the model generator 220, the model selector 230, the respiration signal obtainer 240, the information obtainer 250, the storage 260, and an ultrasound generation 270. These elements are similar to their counterparts found in FIG. 1. The imaging processing apparatus 20, in some embodiments, may further include general-purpose elements other than the elements shown in FIG. 8. Additionally, alternative elements that perform the operation of the imaging processing apparatus 20 may be used instead of the elements shown in FIG. 8.

Also, each of the image generator 210, the model generator 220, the model selector 230, the respiration signal obtainer 240, the information obtainer 250, the storage 260, and the ultrasound generator 270 of the imaging processing apparatus 20 of FIG. 8 may correspond to one or more processors. A processor may include an array of logic gates, or a combination of a general-purpose microprocessor and a program that may be executed by the microprocessor. Alternatively, it would be understood by one of ordinary skill in the art that the processor includes any of other types of hardware.

Operations of the image generator 210, the model generator 220, the model selector 230, the respiration signal obtainer 240, the information obtainer 250, and the storage 260 of the imaging processing apparatus 20 of FIG. 8 are similar to or the same as described above with respect to corresponding elements of FIG. 1.

The ultrasound generator 270 generates diagnostic ultrasound that is to be radiated to a lesion tissue by using obtained information regarding a region of interest 30. That is, if a lesion is present in the region of interest 30, the ultrasound generator 270 generates the diagnostic ultrasound, for example, high-intensity focused ultrasound (HIFU) that is to be radiated by a diagnosis ultrasound probe 60 by using the obtained information regarding the region of interest 30 transmitted from the respiration signal obtainer 240. More specifically, the ultrasound generator 270 may generate a signal that determines conditions of intensity and phase of the diagnosis ultrasound that is to be radiated by elements of the diagnosis ultrasound probe 60. The ultrasound generator 270 transmits the generated signal to the diagnosis ultrasound probe 60.

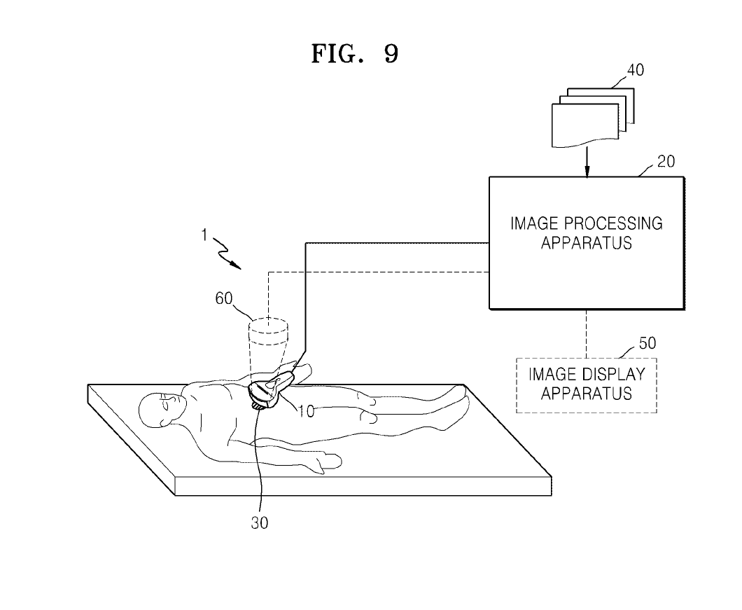

FIG. 9 is a diagram illustrating an environment in which an organ change tracking system 1 is used, according to an embodiment. The organ change tracking system 1 according to an example embodiment includes the diagnosis ultrasound probe 10 and the image processing apparatus 20. The organ change tracking system 1 may further include an image display apparatus 50 or the diagnosis ultrasound probe 60.

The organ change tracking system 1, in some embodiments, may further include general-purpose elements other than the elements shown in FIG. 9. Additionally, alternative elements that perform the operation of the organ change tracking system 1 may be used instead of the elements shown in FIG. 9.

The organ change tracking system 1 of FIG. 9 corresponds to an embodiment of the image processing apparatus 20 of FIGS. 1 and 8. Therefore, the descriptions provided with reference to FIGS. 1 and 8 are also applicable to the organ change tracking system 1 of FIG. 9, and thus redundant descriptions are omitted here.

The diagnosis ultrasound probe 60 radiates HIFU to the lesion present in the region of interest 30. By radiating HIFU to the lesion, as discussed above, the diagnosis ultrasound probe 60 causes ultrasound energy to be focused on the lesion. The focused ultrasound energy becomes heat, which treats the lesion by cauterizing it.

The image display apparatus 50 displays an ultrasound image generated by the image processing apparatus 20. For example, the image display apparatus 50 includes one or more output devices such as a display panel, an LCD screen, and a monitor which are provided in the organ change tracking system 1. Information regarding the region of interest 30 obtained by the image processing apparatus 20 may be provided to a user through the image display apparatus 50 and utilized to determine a status of a tissue or a change in a location or a shape of the tissue. Thus, embodiments provide information that can be used for diagnostic and treatment purposes for lesions or another region of interest 30.

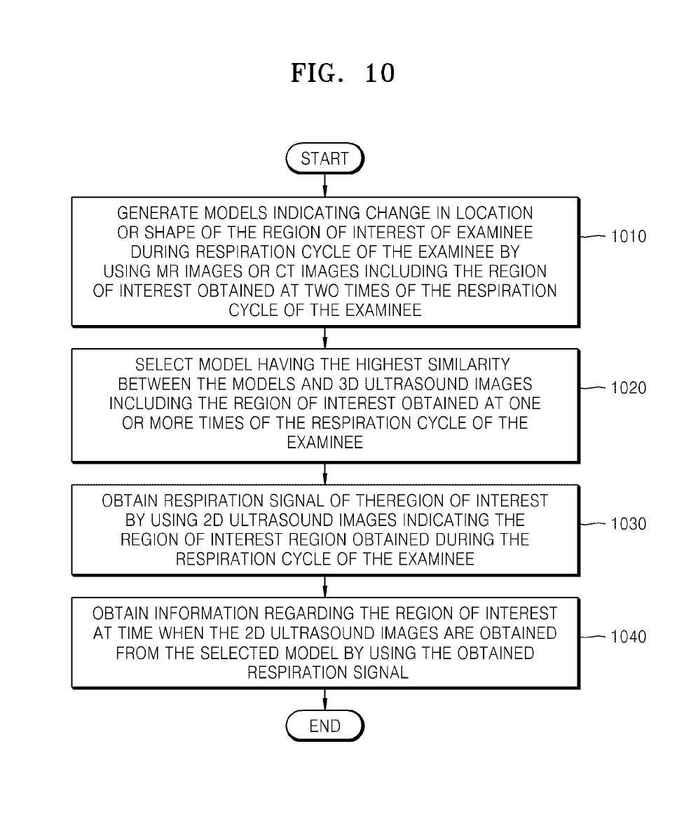

FIG. 10 is a flowchart illustrating a method of tracking a change of an organ during a respiration cycle performed by an image processing apparatus, according to an embodiment. Referring to FIG. 10, the method of tracking the change of the organ includes operations that are time serially performed in the image processing apparatus 20 or the organ change tracking system 1 illustrated in FIGS. 1, 8, and 9. Therefore, although omitted, the above descriptions of the image processing apparatus 20 or the organ change tracking system 1 illustrated in FIGS. 1, 8, and 9 are also relevant to the method of tracking the change of the organ of FIG. 10.

In operation 1010, the model generator 230 generates models indicating a change in a location or a shape of the region of interest 30 during one respiration cycle of a subject by using MR or CT images including the region of interest 30 of the subject obtained at two times of one respiration cycle. While the operation is presented as an embodiment that uses MR or CT images, other types of high-quality images or other images including the region of interest are also usable in different embodiments. In this operation, the two times of one respiration cycle of the subject are maximum inspiration and expiration times of the subject. Additionally, some embodiments use additional images, such as MR or CT images at other times of respiration cycles of the subject, to obtain even better results

In some embodiments, operation 1010 of the model generator 230 may be performed by using MR or CT images obtained before surgery performed on the subject (for example, a patient). For example, the MR or CT images including the region of interest 30 can be gathering during a surgery preparation process on the subject, and the model generator 230 generates models by using the obtained MR or CT images.

In operation 1020, the model generator 230 selects a model having the highest similarity between models and 3D ultrasound images including the region of interest 30 obtained at one or more times of one respiration cycle of the subject. In this regard, the one or more times included for the 3D ultrasound images in some embodiments may be the maximum inspiration time and/or the maximum expiration time during a respiration cycle of the subject.

Operation 1020 of the model generator 230, in an embodiment, may be performed by using 3D ultrasound images generated before surgery performed on the subject. For example, when the subject enters a surgery room and the surgery preparation process ends, the diagnosis ultrasound probe 10 radiates diagnostic ultrasound to the region of interest 30 according to instructions from a user and obtains a reflected ultrasound signal. For example, the user may be a doctor or other health care provider. The image generator 210 generates 3D ultrasound images by using the reflected ultrasound signal. The model selector 230 selects the model having the highest similarity by using the generated 3D ultrasound images.

In operation 1030, the respiration signal obtainer 240 obtains a respiration signal of the region of interest 30 by using obtained 2D ultrasound images included in the region of interest 30 during one respiration cycle of the subject. For example, the respiration signal obtainer 240 may obtain the respiration signal of the region of interest 30 by using 2D ultrasound images transmitted from the image generator 210. In this regard, the respiration signal means a signal indicating a displacement of the region of interest 30 that changes according to the subject's respiration.

The 2D ultrasound images used in operation 1030, in embodiments, may be obtained in real time before and/or during surgery performed on the subject. Operation 1030 of the respiration signal obtainer 240, in embodiments, may be performed before and/or during surgery performed on the subject.

For example, an operation of selecting an object from which the respiration signal is to be obtained and selecting a specific window is performed by the respiration signal obtainer 240 using the 2D ultrasound images obtained before surgery performed on the subject. Meanwhile, an operation of extracting the respiration signal may be performed by the respiration signal obtainer 240 using the 2D ultrasound images obtained in real time during surgery performed on the subject.

In operation 1040, the information obtainer 250 obtains information regarding the region of interest 30 at a time when the 2D ultrasound images are obtained from the selected model by using the obtained respiration signal. In this regard, the time when the 2D ultrasound images are obtained is a time corresponding to one respiration cycle of the subject. The 2D ultrasound images used by the information obtainer 250 are images obtained in real time during surgery performed on the subject.

As described above, the image processing apparatus 20 uses ultrasound images other than X-ray images to track a change in an organ during a respiration cycle of a subject, and thus an image regarding a region of interest may be obtained in real time during surgery, and obtaining such an image is believed harmless to a human body since diagnostic ultrasound may not have negative health effects on a subject. The image processing apparatus 20 tracks a change in an organ by features clearly identified from ultrasound images, and thus noise may be strongly tracked and managed. The image processing apparatus 20 accurately tracks the changes in the organ. By accurately tracking the changes in the organ, a surgery accuracy may be improved and a surgery time may be reduced when applied to HIFU and radiation therapy.

Further, since respiration periodically changes in a predictable way, if locations of organs and lesions according to patient's respiration are known in advance before surgery, current locations of organs and lesions may be estimated by using a respiration signal of a patient in surgery. That is, knowing in advance the configuration of objects in a patient's system and pairing this information with information about predictable, cyclical changes in those objects allow modeling of how organs and lesions will change shape during a particular time period.

The image display apparatus 50 may be implemented as a liquid crystal display (LCD), a light-emitting diode (LED) display, a plasma display panel (PDP), a screen, a terminal, and the like. A screen may be a physical structure that includes one or more hardware components that provide the ability to render a user interface and/or receive user input. The screen can encompass any combination of display region, gesture capture region, a touch sensitive display, and/or a configurable area. The screen can be embedded in the hardware or may be an external peripheral device that may be attached and detached from the apparatus. The display may be a single-screen or a multi-screen display. A single physical screen can include multiple displays that are managed as separate logical displays permitting different content to be displayed on separate displays although part of the same physical screen. The user interface may also be responsible for inputting and outputting input information regarding a user and an image. The interface may include a network module for connection to a network and a universal serial bus (USB) host module for forming a data transfer channel with a mobile storage medium. In addition, the user interface may include an input/output device such as, for example, a mouse, a keyboard, a touch screen, a monitor, a speaker, a screen, and a software module for running the input/output device.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The media may also include, alone or in combination with the software program instructions, data files, data structures, and the like. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

As a non-exhaustive illustration only, a terminal/device/unit described herein may refer to mobile devices such as, for example, a cellular phone, a smart phone, a wearable smart device (such as, for example, a ring, a watch, a pair of glasses, a bracelet, an ankle bracket, a belt, a necklace, an earring, a headband, a helmet, a device embedded in the cloths or the like), a personal computer (PC), a tablet personal computer (tablet), a phablet, a personal digital assistant (PDA), a digital camera, a portable game console, an MP3 player, a portable/personal multimedia player (PMP), a handheld e-book, an ultra mobile personal computer (UMPC), a portable lab-top PC, a global positioning system (GPS) navigation, and devices such as a high definition television (HDTV), an optical disc player, a DVD player, a Blue-ray player, a setup box, or any other device capable of wireless communication or network communication consistent with that disclosed herein. In a non-exhaustive example, the wearable device may be self-mountable on the body of the user, such as, for example, the glasses or the bracelet. In another non-exhaustive example, the wearable device may be mounted on the body of the user through an attaching device, such as, for example, attaching a smart phone or a tablet to the arm of a user using an armband, or hanging the wearable device around the neck of a user using a lanyard.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of tracking a change in a region of interest of a subject according to respiration, comprising:
generating models indicating a change in a location or a shape of the region of interest of the subject during a respiration cycle of the subject by using including the region of interest obtained at two times of the respiration cycle of the subject;
selecting a model having the highest similarity to 3D ultrasound images including the region of interest obtained at one or more times of the respiration cycle of the subject;
obtaining a respiration signal of the region of interest by using 2D ultrasound images including the region of interest obtained during the respiration cycle of the subject; and
obtaining information regarding the region of interest at a time when the 2D ultrasound images are obtained, from the selected model, by using the obtained respiration signal.

2. The method of claim 1, wherein the external images are magnetic resonance (MR) images or computed tomography (CT) images.

3. The method of claim 1, wherein the obtaining of the respiration signal comprises:
selecting an object from which the respiration signal is to be obtained from the 2D ultrasound images;
selecting a specific window from windows disposed in a location indicating the selected object from the 2D ultrasound images; and
generating the respiration signal by using motion information of the object included in the specific window,
wherein the windows have different sizes, directions, and locations disposed on the 2D ultrasound images to obtain the motion information of the object according to the respiration.

4. The method of claim 3, wherein the respiration signal is a signal indicating a displacement of the region of interest that changes according to the subject's respiration.

5. The method of claim 3, wherein the object is an object having a brightness value exceeding a threshold value among organs included in the 2D ultrasound images.

6. The method of claim 3, wherein the selecting of the object comprises:
segmenting information regarding a boundary line of the object from the 2D ultrasound images; and
obtaining a center line of the object by using the segmented information regarding the boundary line,
wherein the specific window is selected by placing the windows on the obtained center line.

7. The method of claim 3, wherein the specific window is selected by using at least one of noise information of the 2D ultrasound images or the motion information of the object.

8. The method of claim 1, wherein the two times are maximum inspiration time and maximum expiration time of the subject.

9. The method of claim 8, wherein the generating of the models comprises:
segmenting surface information of tissues included in the external images obtained at the maximum inspiration time and the external images obtained at the maximum expiration time; and
performing interpolation by using the segmented surface information.

10. The method of claim 1, wherein the selecting of the model comprises:
segmenting surface information of tissues included in the 3D ultrasound images;
matching the models and the 3D ultrasound images by using the segmented surface information; and
calculating similarity between the models and the 3D ultrasound images by using the matching images and selecting a model having the highest similarity between the models and the 3D ultrasound images by using the calculated similarity.

11. The method of claim 1, wherein the obtaining of the information comprises: obtaining information regarding the region of interest by using at least one of a displacement value of the region of interest at the time when the 2D ultrasound images are obtained and maximum and minimum values of the displacement value of the region of interest included in the selected model,
wherein the time when the 2D ultrasound images are obtained comprises a time of the respiration cycle of the subject.

12. The method of claim 1, further comprising: generating ultrasound that is to be radiated to the lesion tissue by using the obtained information regarding the region of interest.

13. A non-transitory computer-readable storage medium storing a program for tracking a change in a region of interest, the program comprising instructions for causing a computer to carry out the method of claim 1.

14. An apparatus for tracking a change in a region of interest of a subject according to respiration, comprising:
a model generator configured to generate models indicating a change in a location or a shape of the region of interest of the subject during a respiration cycle of the subject by using external images including the region of interest obtained at two times of the respiration cycle of the subject;
a model selector configured to select a model having the highest similarity between the models and 3D ultrasound images including the region of interest obtained at one or more times of the respiration cycle of the subject;
a respiration signal obtainer configured to obtain a respiration signal of the region of interest by using 2D ultrasound images indicating the region of interest obtained during the respiration cycle of the subject; and
an information obtainer configured to obtain information regarding the region of interest at a time when the 2D ultrasound images are obtained, from the selected model, by using the obtained respiration signal.

15. The apparatus of claim 14, wherein the external images are magnetic resonance (MR) images or computed tomography (CT) images.

16. The apparatus of claim 14, wherein the respiration signal obtainer is configured to select an object from which the respiration signal is to be obtained from the 2D ultrasound images, configured to select a specific window from windows disposed in a location indicating the selected object from the 2D ultrasound images, and configured to generate the respiration signal by using motion information of the object included in the specific window,
wherein the windows have different sizes, directions, and locations disposed on the 2D ultrasound images to obtain the motion information of the object according to the respiration.

17. The apparatus of claim 16, wherein the object is selected by segmenting information regarding a boundary line of the object from the 2D ultrasound images, and obtaining a center line of the object by using the segmented information regarding the boundary line,
wherein the specific window is selected by placing the windows on the obtained center line.

18. The apparatus of claim 14, wherein the model generator is configured to segment surface information of tissues included in the external images obtained at two times of the respiration cycle of the subject and configured to perform interpolation by using the segmented surface information,
wherein the two times are maximum inspiration time and maximum expiration time of the subject.

19. The apparatus of claim 14, wherein the model selector is configured to segment surface information of tissues included in the 3D ultrasound images, configured to match the models and the 3D ultrasound images by using the segmented surface information, configured to calculate similarity between the models and the 3D ultrasound images by using the matching images, and configured to select a model having the highest similarity between the models and the 3D ultrasound images by using the calculated similarity.

20. The apparatus of claim 14, further comprising: an ultrasound generator configured to generate diagnosis ultrasound that is to be radiated to the lesion tissue by using the obtained information regarding the region of interest.
